(19) United States
(12) Patent Application Publication
(10) Pub. No.: US 2005/0159602 A1
(43) Pub. Date: Jul. 21, 2005

(54) Method for synthesizing chiral bicyclic thiazolidine hydantoin

(75) Inventors: Chih-Wei Kuo, Tali City (TW); Ko-Chieh Ho, Hualien City (TW); Rai-Yi Chen, Pingchen City (TW); Yu-Ting Shen, Tainan Hsien (TW); Ying-Jen Hung, Changhua Hsien (TW); Ling Lu, Taoyuan Hsien (TW)

Correspondence Address: Dennison, Schultz & Dougherty & MacDonald, Suite 105, 1727 King Street, Alexandria, VA, 22314, US

(73) Assignee: LABELTEK INC.

(21) Appl. No.: 10/758,223

(22) Filed: Jan. 16, 2004

(51) Int. Cl. C07D 498/04

(52) U.S. Cl. 548/154

(57) ABSTRACT

A method for synthesizing chiral bicyclic thiazolidine hydantoin that uses L-(+)-Cysteine, an aldehyde, and a preferred benzylisocyanate as reactants with additive solid molecular sieves to efficiently synthesize chiral bicyclic thiazolidine hydantoin crystallization having high purity. This method can be operated within only a singular reacting chamber without isolating intermediates in this method. Thereby, operational procedures of the present invention are simplified to make the method economic.

### BACKGROUND OF THE INVENTION

1. Field of the Invention

The present invention relates to a method for synthesizing chiral bicyclic thiazolidine hydantoin, and more particularly to a method that synthesizes chiral bicyclic thiazolidine hydantoin having high purity but by using only one reacting chamber.

2. Description of Related Art

Biochips are used to obtain biotic information and each biochip basically composes a substrate made of glass or a Nylon membrane and multiple probes made of single-strand DNA, protein, antigen, or antibody attached to the substrate. The probes hybridize with target sequences and are processed to generate signals such as luminescent spots. Then, the signals are analyzed, compared with a built-in database, judged, and translated to become useful bio-information. The biochips are classified into three types by different characteristics, which are gene-chips (i.e. DNA microarray), lab-on-a-chips, and protein-chips. Wherein, the gene-chip has several advantages of rapid screening, precise detection and mass sample sieving. Thus, in the post-gene age, the biochips improve the progress of automatic gene analysis and are the most prominent products in biotech industry.

Bioactive bicyclic thiazolidine hydantoin is a key intermediate to synthesize biotin that is a labeling material applied on a protein or a DNA sequence. Prior patents U.S. Pat. No. 4,009,172, U.S. Pat. No. 4,130,713, U.S. Pat. No. 4,337,345, U.S. Pat. No. 4,550,075, U.S. Pat. No. 4,732,987, U.S. Pat. No. 4,837,402, U.S. Pat. No. 4,877,882, U.S. Pat. No. 4,937,351, U.S. Pat. No. 5,250,699, U.S. Pat. No. 506,834, U.S. Pat. No. 5,095,118 and EP0243734 disclosed several methods for preparing biotin by bicyclic thiazolidine hydantoin, but rarely mentioned methods for synthesizing bicyclic thiazolidine hydantoin.

According to records of Chem. Ber. 1948, Vol. 81, p210 and Tetrahedron Lett. 1988, Vol. 29, p57, a method for synthesizing bicyclic thiazolidine hydantoin is to use L-cystine dimethyl ester dihydrochloride as a reactant to react with proper chemical agents. The synthesizing reaction in the method is shown as follows:

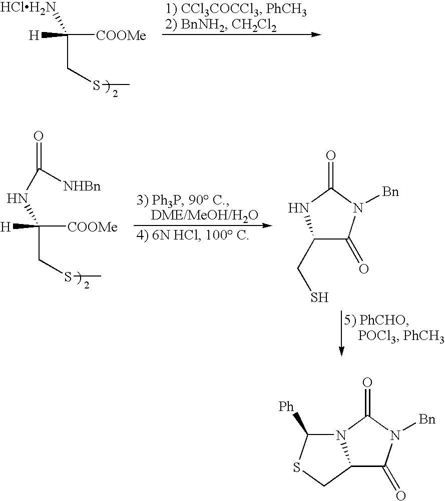

However, the foregoing method in the records has the following drawbacks:
1. The synthesizing reaction takes an excessive amount of time since there are 5 steps in the reaction.
2. The chemical agents are expansive and have toxicity.
3. Other waste products such as triphenylphosphine oxide, are difficult to treat, even in a furnace.
4. It is difficult to crystallize bicyclic thiazolidine hydantoin.
5. The purifying process of bicyclic thiazolidine hydantoin is complex.

Based on those drawbacks, the foregoing method is not suitable for industrial manufacturing.

Another method for synthesizing bicyclic thiazolidine hydantoin is disclosed in records of Chimia 1987, Vol. 41, p148 and the Journal of Organic Chemistry 1955, Vol. 60, p320-321, wherein L-(+)-Cysteine is reacted with benzyl aldehyde to generate 4(R)-carboxy-2-phenylthiazolidine in a first cycloaddition reaction. Then, the generated 4(R)-carboxy-2-phenylthiazolidine is purified and further mixed with benzylisocyanate to compose bicyclic thiazolidine hydantoin in a second cycloaddition reaction. However, this method still has the following drawbacks:
1. Cycloaddition reactions and purifying processes in this method have to be separately carried out which causes a low product rate and unstable quality of bicyclic thiazolidine hydantoin.
2. It is difficult to crystallize bicyclic thiazolidine hydantoin.
3. This method requires an excessive amount of time to carry out the separately multiple cycloaddition reactions and purifying processes.

Therefore, this method is also not suitable for industrial manufacturing.

The present invention has arisen to provide a method for synethesizing bicyclic thiazolidine hydantoin to overcome and obviate the drawbacks of the conventional methods.

### SUMMARY OF THE INVENTION

A first objective of the present invention is to provide a method for synthesizing bicyclic thiazolidine hydantoin, which carries out cycloadditions twice in only one reacting chamber (one-pot) to conveniently generate bicyclic thiazolidine hydantoin.

A second objective of the present invention is to provide a method for synthesizing bicyclic thiazolidine hydantoin, which does not need to isolate intermediate during operating so that the method is simplified to save manufacturing costs, and eased from treating waste generated in an isolating process.

A third objective of the present invention is to provide a method for synthesizing bicyclic thiazolidine hydantoin, which shortens reaction time and simplifies a purifying process to avoid washing bicyclic thiazolidine hydantoin away.

A fourth objective of the present invention is to provide a method for synthesizing bicyclic thiazolidine hydantoin, which adds molecular sieves to remove water from the cycloaddition reactions to increase the crystallization degree of bicyclic thiazolidine hydantoin, wherein the molecular sieves can be recycled.

Further benefits and advantages of the present invention will become apparent after a careful reading of the detailed description in company with the drawings.

### BRIEF DESCRIPTION OF THE DRAWINGS

FIGS. 1A-1B are schematic flowcharts of a method for synthesizing bicyclic thiazolidine hydantoin in accordance with the present invention, wherein the method is carried out within only one reacting chamber.

### DETAILED DESCRIPTION OF THE PREFERRED EMBODIMENT

A method for synthesizing chiral bicyclic thiazolidine hydantoin in accordance with the present invention comprises the following chemical equation: 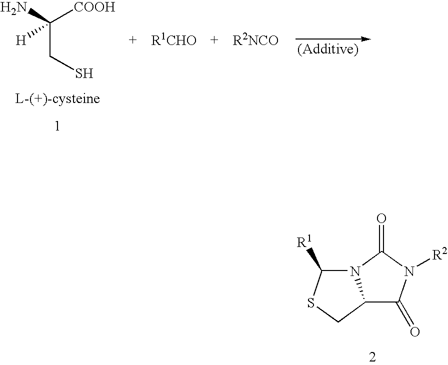
wherein R¹ and R² are selected from the group comprising a hydrogen, phenyl, benzyl, alkyl group containing 1-5 carbon atoms, aryl alkyl group in which the alkyl containing 1-5 carbon atoms.

Wherein R¹ is preferred to be phenyl and R² is preferred to be benzyl.

The chemical equation in the present invention basically has two main reactions:
(1) L-(+)-Cysteine (compound 1) and an aldehyde are reactants and dissolved in an organic alcohol solvent to carry out a first cycloaddition to generate white intermediate, 4(R)-carboxy-2-phenylthiazolidine.
(2) Solid molecular sieves and preferred benzylisocyanate are added into 4(R)-carboxy-2-phenylthiazolidine to carry out a second cycloaddition to generate bicyclic thiazolidine hydantoin. Lastly, an alcohol solvent is added into the reaction to accelerate crystallization of bicyclic thiazolidine hydantoin (compound 2).

In the second cycloaddition, a ketone solvent and an ester solvent are respectively used to dissolve the reactants.

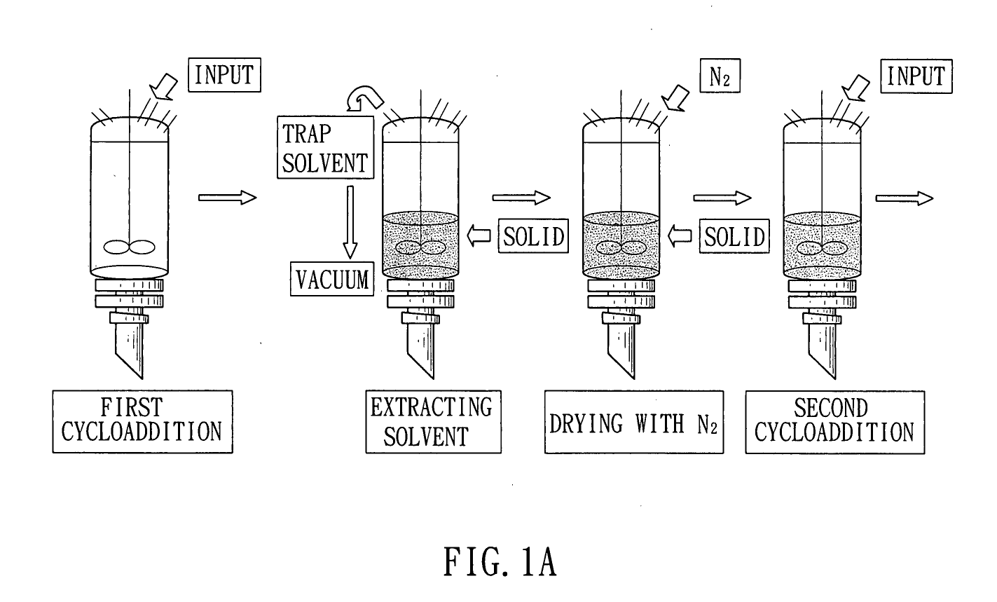

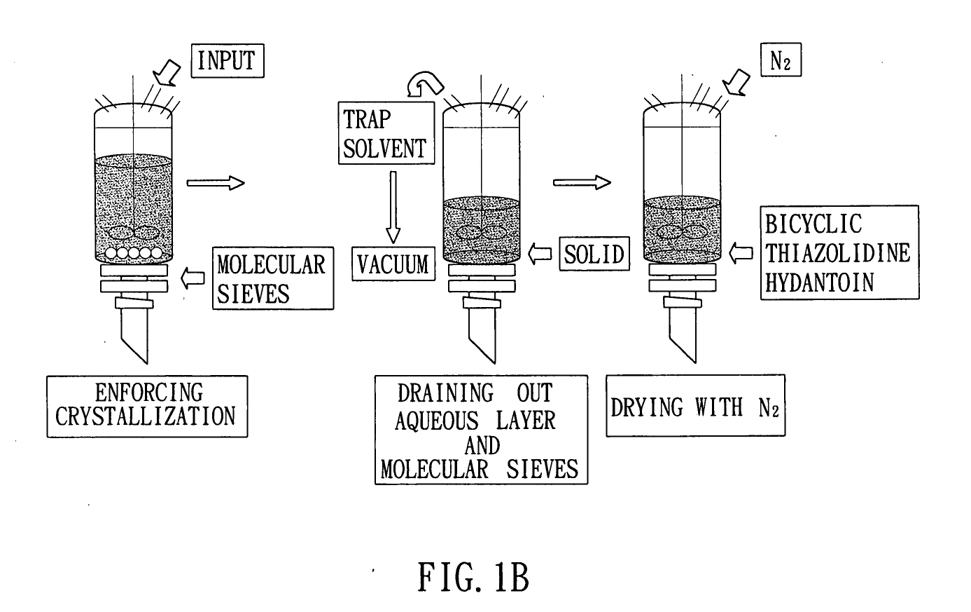

With reference to FIGS. 1A and 1B, a detailed description of the method for synthesizing bicyclic thiazolidine hydantoin is illustrated in accordance with the drawings. L-(+)-Cysteine (compound 1) and an aldehyde or derivatives of the aldehyde are initial reactants and inputted into a reacting chamber with a bottom as shown in the drawings. An organic alcohol-water solvent (water:organic alcohol=1:1) and an organic alkali are conducted into the reacting chamber. The first cycloaddition occurs to last for 2 hours at room temperature (25° C.) to generate white solid intermediate, 4(R)-carboxy-2-phenylthiazolidine. The organic alcohol-water solvent is trapped out by a vacuum system and then 4(R)-carboxy-2-phenylthiazolidine is dried by blowing nitrogen gas. Preferred benzylisocyanate, or derivatives of isocyanate are dissolved in a ketone solvent and introduced into the reacting chamber to react with the intermediate in the second cycloaddition, wherein the ketone contains 2 to 5 carbon atoms. The second cycloaddition occurs to last for 2 hours at 25 to 30° C. Then, the ketone solvent is extracted out by the vacuum system. An ester solvent or an ether solvent and an inorganic acid are added into the reacting chamber to achieve a mixed solution, wherein alkyl of the ester or the ether contains 1 to 4 carbon atoms. Then, solid molecular sieves are inputted into the mixed solution. The mixed solution is stirred for 2 hours at 25° C. to 30° C. After stirring, the mixed solution is steady placed to make the mixed solution separate into two layers, one is an upper ester layer or an upper ether at the top and the other is an aqueous layer with the solid molecular sieves at the bottom. The aqueous layer and the solid molecular sieves are drained out of the reacting chamber via an outlet at the bottom of the reacting chamber. The ester solvent or ether solvent is extracted out by the vacuum system. Then, an alcohol solvent containing 1 to 4 carbon atoms is added into the reacting chamber to enforce crystallization of bicyclic thiazolidine hydantoin within 10 minutes. Again, the vacuum system is operated to remove the residual alcohol solvent. Lastly, crystallization of bicyclic thiazolidine hydantoin is dried in a vacuum drying apparatus to obtain final white solid bicyclic thiazolidine hydantoin (compound 2). Thereby, the first and second cycloadditions are realized in a singular reacting chamber to synthesize bicyclic thiazolidine hydantoin.

The organic alkali is sodium acetate or potassium acetate. The solid molecular sieves are in the form of particles having 3 Å-to 5 Å bore diameters. The ether having 1 to 4 carbons is preferred to be diethyl ether. The ester having 1 to 4 carbons is selected from the group comprising methyl formate, ethyl formate, methyl acetate, ethyl acetate, and propyl acetate. The inorganic acid is 6N hydrochloric acid.

The reacting chamber shown in FIGS. 1A and 1B is designed to carry out synthesis, and filtering processes without isolating intermediate in this method. Additionally, the solvents are extracted out by the vacuum system after the first cycloaddition reaction to allow directly operating the second cycloaddition reaction without changing the reacting chamber. Therefore, operational procedures of the present invention are simplified so that synthesizing efficiency is increased and product loss is decreased.

Moreover, the solid molecular sieves are added into the reaction to remove water and to increase the crystallizing rate of bicyclic thiazolidine hydantoin. The solid molecular sieves are enabled to be recycled and reused to decrease waste in this method.

The following examples are shown to further illustrate details in the present invention.

### EXAMPLE 1

Initially, 1000 g of L-(+)-Cysteine (8.3 moles), 880 g of benzyl aldehyde (8.3 moles), 750 g of sodium acetate (9.1 moles) were introduced into a 10 L reacting chamber. Then, 4 L of water and 4 L of methanol were poured into the reacting chamber to dissolve the chemicals to become a solution. The solution was stirred for 2 hours at 25° C. and then a white solid appeared in the solution. The white solid was examined and determined as of 4(R)-carboxy-2-phenylthiazolidine (see the Appendix 1, NMR.hydrogen spectrum). Nitrogen gas was introduced to flow through the reacting chamber and lasted for 30 minutes to remove residual methanol. Then, 500 g of 3 Å molecular sieves and 1320 g of benzylisocyanate (9.9 moles) were added into the solution. Next, 5 L of acetone was conducted into the solution. The solution was stirred for 2 hours at 25° C. After stirring, the acetone was extracted out of the solution by a vacuum extracting device. Then, 3 L of ethyl acetate and 3 L of 6N hydrochloric acid were added into the solution. Again, the solution was stirred for 2 hours at 25° C. and stably placed for 10 minutes until the solution was separated into an upper ethyl acetate layer and a lower aqueous layer with deposited molecular sieves. The lower aqueous layer and the deposited molecular sieves were drained out of the reacting chamber. The ethyl acetate layer remaining in the reacting chamber was extracted by the vacuum extracting device to remove the ethyl acetate. Within 10 minutes, 4 L of methanol was introduced into the reacting chamber to enforce crystallization of bicyclic thiazolidine hydantoin in the form of a white solid. Residual methanol was extracted from the white solid by nitrogen gas flowing through the reacting chamber. Lastly, the bicyclic thiazolidine hydantoin was dried in a vacuum drying apparatus at 20° C. for 3 hours to obtain a final product, 2522 g of bicyclic thiazolidine hydantoin (86% producing rate), having a melting point at 79 to 80° C. [a]_{D}²⁰=−279.83′ C=1(CH₂Cl₂).

### EXAMPLE 2

Initially, 1000 g of L-(+)-Cysteine (8.3 moles), 880 g of benzyl aldehyde (8.3 moles), 750 g of sodium acetate (9.1 moles) were introduced into a 10 L reacting chamber. Then, 4 L of water and 4 L of methanol were poured into the reacting chamber to dissolve the chemicals to become a solution. The solution was stirred for 2 hours at 25° C. and then a white solid appeared in the solution. The white solid was examined and determined as of 4(R)-carboxy-2-phenylthiazolidine. Nitrogen gas was introduced to flow through the reacting chamber and lasted for 30 minutes to remove residual methanol. Then, 500 g of 4 Å molecular sieves and 1320 g of benzylisocyanate (9.9 moles) were added into the solution. Next, 5 L of acetone was conducted into the solution. The solution was stirred for 2 hours at 25° C. After stirring, the acetone was extracted out of the solution by a vacuum extracting device. Then, 3 L of ethyl acetate and 3 L of 6N hydrochloric acid were added into the solution. Again, the solution was stirred for 2 hours at 25° C. and stably placed for 10 minutes until the solution was separated into an upper ethyl acetate layer and a lower aqueous layer with deposited 4 Å molecular sieves. The lower aqueous layer and the deposited 4 Å molecular sieves were drained out of the reacting chamber. The ethyl acetate layer remaining in the reacting chamber was extracted by the vacuum extracting device to remove the ethyl lacetate. Within 10 minutes, 4 L of methanol was introduced into the reacting chamber to enforce crystallization of bicyclic thiazolidine hydantoin in the form of a white solid. Residual methanol was extracted from the white solid by nitrogen gas flowing through the reacting chamber. Lastly, the bicyclic thiazolidine hydantoin was dried in a vacuum drying apparatus at 20° C. for 3 hours to obtain a final product, 2347 g of bicyclic thiazolidine hydantoin (80% producing rate), having a melting point at 80° C. [a]_{D}²⁰=−288.44′ C=1(CH₂Cl₂).

### EXAMPLE 3

Initially, 1000 g of L-(+)-Cysteine (8.3 moles), 880 g of benzyl aldehyde (8.3 moles), 750 g of sodium acetate (9.1 moles) were introduced into a 10 L reacting chamber. Then, 4 L of water and 4 L of methanol were poured into the reacting chamber to dissolve the chemicals to become a solution. The solution was stirred for 2 hours at 25° C. and then a white solid appeared in the solution. The white solid was examined and determined as of 4(R)-carboxy-2-phenylthiazolidine. Nitrogen gas was introduced to flow through the reacting chamber and lasted for 30 minutes to remove residual methanol. Then, 500 g of 5 Å molecular sieves and 1320 g of benzylisocyanate (9.9 moles) were added into the solution. Next, 5 L of acetone was conducted into the solution. The solution was stirred for 2 hours at 25° C. After stirring, the acetone was extracted out of the solution by a vacuum extracting device. Then, 3 L of ethyl acetate and 3 L of 6N hydrochloric acid were added into the solution. Again, the solution was stirred for 2 hours at 25° C. and stably placed for 10 minutes until the solution was separated into an upper ethyl acetate layer and a lower aqueous layer with deposited 5 Å molecular sieves. The lower aqueous layer and the deposited 5 Å molecular sieves were drained out of the reacting chamber. The ethyl acetate layer remaining in the reacting chamber was extracted by the vacuum extracting device to remove the ethyl acetate. Within 10 minutes, 4 L of methanol was introduced into the reacting chamber to enforce crystallization of bicyclic thiazolidine hydantoin in the form of a white solid. Residual methanol was extracted from the white solid by nitrogen gas flowing through the reacting chamber. Lastly, the bicyclic thiazolidine hydantoin was dried in a vacuum drying apparatus at 20° C. for 3 hours to obtain a final product, 2200 g of bicyclic thiazolidine hydantoin (75% producing rate), having a melting point at 79 to 80° C. [a]_{D}²⁰=−280.36′ C=1(CH₂Cl₂).

### EXAMPLE 4

Initially, 1000 g of L-(+)-Cysteine (8.3 moles), 880 g of benzyl aldehyde (8.3 moles), 750 g of sodium acetate (9.1 moles) were introduced into a 10 L reacting chamber. Then, 4 L of water and 4 L of ethanol were poured into the reacting chamber to dissolve the chemicals to become a solution. The solution was stirred for 2 hours at 25° C. and then a white solid appeared in the solution. The white solid was examined and determined as of 4(R)-carboxy-2-phenylthiazolidine. Nitrogen gas was introduced to flow through the reacting chamber and lasted for 30 minutes to remove residual ethanol. Then, 500 g of 3 Å molecular sieves and 1320 g of benzylisocyanate (9.9 moles) were added into the solution. Next, 5 L of acetone was conducted into the solution. The solution was stirred for 2 hours at 25° C. After stirring, the acetone was extracted out of the solution by a vacuum extracting device. Then, 3 L of ethyl acetate and 3 L of 6N hydrochloric acid were added into the solution. Again, the solution was stirred for 2 hours at 25° C. and stably placed for 10 minutes until the solution was separated into an upper ethyl acetate layer and a lower aqueous layer with deposited 3 Å molecular sieves. The lower aqueous layer and the deposited 3 Å molecular sieves were drained out of the reacting chamber. The ethyl acetate layer remaining in the reacting chamber was extracted by the vacuum extracting device to remove the ethyl acetate. Within 10 minutes, 4 L of methanol was introduced into the reacting chamber to enforce crystallization of bicyclic thiazolidine hydantoin in the form of a white solid. Residual methanol was extracted from the white solid by nitrogen gas flowing through the reacting chamber. Lastly, the bicyclic thiazolidine hydantoin was dried in a vacuum drying apparatus at 20° C. for 3 hours to obtain a final product, 2405 g of bicyclic thiazolidine hydantoin (82% producing rate), having a melting point at 80° C. [a]_{D}²⁰=−280.59′ C=1(CH₂Cl₂).

### EXAMPLE 5

Initially, 1000 g of L-(+)-Cysteine (8.3 moles), 880 g of benzyl aldehyde (8.3 moles), 750 g of sodium acetate (9.1 moles) were introduced into a 10 L reacting chamber. Then, 4 L of water and 4 L of methanol were poured into the reacting chamber to dissolve the chemicals to become a solution. The solution was stirred for 2 hours at 25° C. and then a white solid appeared in the solution. The white solid was examined and determined as of 4(R)-carboxy-2-phenylthiazolidine. Nitrogen gas was introduced to flow through the reacting chamber and lasted for 30 minutes to remove residual methanol. Then, 500 g of 3 Å molecular sieves and 1320 g of benzylisocyanate (9.9 moles) were added into the solution. Next, 5 L of acetone was conducted into the solution. The solution was stirred for 2 hours at 25° C. After stirring, the acetone was extracted out of the solution by a vacuum extracting device. Then, 3 L of diethyl ether and 3 L of 6N hydrochloric acid were added into the solution. Again, the solution was stirred for 2 hours at 25° C. and stably placed for 10 minutes until the solution was separated into an upper diethyl ether layer and a lower aqueous layer with deposited 3 Å molecular sieves. The lower aqueous layer and the deposited 3 Å molecular sieves were drained out of the reacting chamber. The diethyl ether layer remaining in the reacting chamber was extracted by the vacuum extracting device to remove the diethyl ether. Within 10 minutes, 4 L of ethanol was introduced into the reacting chamber to enforce crystallization of bicyclic thiazolidine hydantoin in the form of a white solid. Residual ethanol was extracted from the white solid by nitrogen gas flowing through the reacting chamber. Lastly, the bicyclic thiazolidine hydantoin was dried in a vacuum drying apparatus at 20° C. for 3 hours to obtain a final product, 1995 g of bicyclic thiazolidine hydantoin (68% producing rate), having a melting point at 80° C. [a]_{D}²⁰=−280.36′ C=1(CH₂Cl₂).

### EXAMPLE 6

Initially, 1000 g of L-(+)-Cysteine (8.3 moles), 880 g of benzyl aldehyde (8.3 moles), 750 g of sodium acetate (9.1 moles) were introduced into a 10 L reacting chamber. Then, 4 L of water and 4 L of ethanol were poured into the reacting chamber to dissolve the chemicals to become a solution. The solution was stirred for 2 hours at 25° C. and then a white solid appeared in the solution. The white solid was examined and determined as of 4(R)-carboxy-2-phenylthiazolidine. Nitrogen gas was introduced to flow through the reacting chamber and lasted for 30 minutes to remove residual ethanol. Then, 500 g of 3 Å molecular sieves and 1320 g of benzylisocyanate (9.9 moles) were added into the solution. Next, 5 L of acetone was conducted into the solution. The solution was stirred for 2 hours at 25° C. After stirring, the acetone was extracted out of the solution by a vacuum extracting device. Then, 3 L of ethyl acetate and 3 L of 6N hydrochloric acid were added into the solution. Again, the solution was stirred for 2 hours at 25° C. and stably placed for 10 minutes until the solution was separated into an upper ethyl acetate layer and a lower aqueous layer with deposited 3 Å molecular sieves. The lower aqueous layer and the deposited 3 Å molecular sieves were drained out of the reacting chamber. The ethyl acetate layer remaining in the reacting chamber was extracted by the vacuum extracting device to remove the ethyl acetate. Within 10 minutes, 4 L of isopropanol was introduced into the reacting chamber to enforce crystallization of bicyclic thiazolidine hydantoin in the form of a white solid. Residual isopropanol was extracted from the white solid by nitrogen gas flowing through the reacting chamber. Lastly, the bicyclic thiazolidine hydantoin was dried in a vacuum drying apparatus at 20° C. for 3 hours to obtain a final product, 2552 g of bicyclic thiazolidine hydantoin (87% producing rate), having a melting point at 79 to 80° C. [a]_{D}²⁰=−280.3′ C=1(CH₂Cl₂).

### EXAMPLE 7

Initially, 100 g of L-(+)-Cysteine (8.3 moles), 880 g of benzyl aldehyde (8.3 moles), 750 g of sodium acetate (9.1 moles) were introduced into a 10 L reacting chamber. Then, 4 L of water and 4 L of isopropanol were poured into the reacting chamber to dissolve the chemicals to become a solution. The solution was stirred for 2 hours at 25° C. and then a white solid appeared in the solution. The white solid was examined and determined as of 4(R)-carboxy-2-phenylthiazolidine. Nitrogen gas was introduced to flow through the reacting chamber and lasted for 30 minutes to remove residual isopropanol. Then, 500 g of 3 Å molecular sieves and 1320 g of benzylisocyanate (9.9 moles) were added into the solution. Next, 5 L of acetone was conducted into the solution. The solution was stirred for 2 hours at 25° C. After stirring, the acetone was extracted out of the solution by a vacuum extracting device. Then, 3 L of ethyl acetate and 3 L of 6N hydrochloric acid were added into the solution. Again, the solution was stirred for 2 hours at 25° C. and stably placed for 10 minutes until the solution was separated into an upper ethyl acetate layer and a lower aqueous layer with deposited 3 Å molecular sieves. The lower aqueous layer and the deposited 3 Å molecular sieves were drained out of the reacting chamber. The ethyl acetate layer remaining in the reacting chamber was extracted by the vacuum extracting device to remove the ethyl acetate. Within 10 minutes, 4 L of methanol was introduced into the reacting chamber to enforce crystallization of bicyclic thiazolidine hydantoin in the form of a white solid. Residual methanol was extracted from the white solid by nitrogen gas is flowing through the reacting chamber. Lastly, the bicyclic thiazolidine hydantoin was dried in a vacuum drying apparatus at 20° C. for 3 hours to obtain a final product, 2347 g of bicyclic thiazolidine hydantoin (80% producing rate), having a melting point at 80° C. [a]_{D}²⁰=−280.01′ C=1(CH₂Cl₂).

### EXAMPLE 8

Initially, 1000 g of L-(+)-Cysteine (8.3 moles), 880 g of benzyl aldehyde (8.3 moles), 750 g of sodium acetate (9.1 moles) were introduced into a 10 L reacting chamber. Then, 4 L of water and 4 L of isopropanol were poured into the reacting chamber to dissolve the chemicals to become a solution. The solution was stirred for 2 hours at 25° C. and then a white solid appeared in the solution. The white solid was examined and determined as of 4(R)-carboxy-2-phenylthiazolidine. Nitrogen gas was introduced to flow s through the reacting chamber and lasted for 30 minutes to remove residual isopropanol. Then, 500 g of 3 Å molecular sieves and 1320 g of benzylisocyanate (9.9 moles) were added into the solution. Next, 5 L of acetone was conducted into the solution. The solution was stirred for 2 hours at 25° C. After stirring, the acetone was extracted out of the solution by a vacuum extracting device. Then, 3 L of ethyl acetate and 3 L of 6N hydrochloric acid were added into the solution. Again, the solution was stirred for 2 hours at 25° C. and stably placed for 10 minutes until the solution was separated into an upper ethyl acetate layer and a lower aqueous layer with deposited 3 Å molecular sieves. The lower aqueous layer and the deposited 3 Å molecular sieves were drained out of the reacting chamber. The ethyl acetate layer remaining in the reacting chamber was extracted by the vacuum extracting device to remove the ethyl acetate. Within 10 minutes, 4 L of ethanol was introduced into the reacting chamber to enforce crystallization of bicyclic thiazolidine hydantoin in the form of a white solid. Residual ethanol was extracted from the white solid by nitrogen gas flowing through the reacting chamber. Lastly, the bicyclic thiazolidine hydantoin was dried in a vacuum drying apparatus at 20° C. for 3 hours to obtain a final product, 2376 g of bicyclic thiazolidine hydantoin (81% producing rate), having a melting point at 79 to 80° C. [a]_{D}²⁰=−280.59′ C=1(CH₂Cl₂).

### EXAMPLE 9

Initially, 1000 g of L-(+)-Cysteine (8.3 moles), 880 g of benzyl aldehyde (8.3 moles), 750 g of sodium acetate (9.1 moles) were introduced into a 10 L reacting chamber. Then, 4 L of water and 4 L of isopropanol were poured into the reacting chamber to dissolve the chemicals to become a solution. The solution was stirred for 2 hours at 25° C. and then a white solid appeared in the solution. The white solid was examined and determined as of 4(R)-carboxy-2-phenylthiazolidine. Nitrogen gas was introduced to flow through the reacting chamber and lasted for 30 minutes to remove residual isopropanol. Then, 500 g of 3 Å molecular sieves and 1320 g of benzylisocyanate (9.9 moles) were added into the solution. Next, 5 L of acetone was conducted into the solution. The solution was stirred for 2 hours at 25° C. After stirring, the acetone was extracted out of the solution by a vacuum extracting device. Then, 3 L of ethyl acetate and 3 L of 6N hydrochloric acid were added into the solution. Again, the solution was stirred for 2 hours at 25° C. and stably placed for 10 minutes until the solution was separated into an upper ethyl acetate layer and a lower aqueous layer with deposited 3 Å molecular sieves. The lower aqueous layer and the deposited 3 Å molecular sieves were drained out of the reacting chamber. The ethyl acetate layer remaining in the reacting chamber was extracted by the vacuum extracting device to remove the ethyl acetate. Within 10 minutes, 4 L of methanol was introduced into the reacting chamber to enforce crystallization of bicyclic thiazolidine hydantoin in the form of a white solid. Residual methanol was extracted from the white solid by nitrogen gas flowing through the reacting chamber. Lastly, the bicyclic thiazolidine hydantoin was dried in a vacuum drying apparatus at 20° C. for 3 hours to obtain a final product, 2464 g of bicyclic thiazolidine hydantoin (84% producing rate), having a melting point at 80° C. [a]_{D}²⁰=−280.10′ C=1(CH₂Cl₂).

### EXAMPLE 10

Initially, 1000 g of L-(+)-Cysteine (8.3 moles), 880 g of benzyl aldehyde (8.3 moles), 750 g of sodium acetate (9.1 moles) were introduced into a 10 L reacting chamber. Then, 4 L of water and 4 L of methanol were poured into the reacting chamber to dissolve the chemicals to become a solution. The solution was stirred for 2 hours at 25° C. and then a white solid appeared in the solution. The white solid was examined and determined as of 4(R)-carboxy-2-phenylthiazolidine. Nitrogen gas was introduced to flow through the reacting chamber and lasted for 30 minutes to remove residual methanol. Then, 500 g of 3 Å molecular sieves and 1320 g of benzylisocyanate (9.9 moles) were added into the solution. Next, 5 L of acetone was conducted into the solution. The solution was stirred for 2 hours at 25° C. After stirring, the acetone was extracted out of the solution by a vacuum extracting device. Then, 3 L of ethyl acetate and 3 L of 6N hydrochloric acid were added into the solution. Again, the solution was stirred for 2 hours at 25° C. and stably placed for 10 minutes until the solution was separated into an upper ethyl acetate layer and a lower aqueous layer with deposited 3 Å molecular sieves. The lower aqueous layer and the deposited 3 Å molecular sieves were drained out of the reacting chamber. The ethyl acetate layer remaining in the reacting chamber was extracted by the vacuum extracting device to remove the ethyl acetate. Within 10 minutes, 4 L of isopropanol was introduced into the reacting chamber to enforce crystallization of bicyclic thiazolidine hydantoin in the form of a white solid. Residual isopropanol was extracted from the white solid by nitrogen gas flowing through the reacting chamber. Lastly, the bicyclic thiazolidine hydantoin was dried in a vacuum drying apparatus at 20° C. for 3 hours to obtain a final product, 2493 g of bicyclic thiazolidine hydantoin (85% producing rate), having a melting point at 78 to 79° C. [a]_{D}²⁰=−279.59′ C=1(CH₂Cl₂).

According to the foregoing examples, each example obtains a high-purity white solid of bicyclic thiazolidine hydantoin that is tested for analysis by NMR and HPLC and shown in the Appendixes 3 to 5. Appendixes 1 and is 2 are spectrums respectively representing ¹H standard test and ¹³C standard test to clarify the intermediate 4(R)-carboxy-2-phenylthiazolidine. Appendixes 3 and 4 are spectrums respectively representing ¹H standard test and ¹³C standard test to clarify the final product, bicyclic thiazolidine hydantoin. Appendix 5 is an HPLC testing result to show the purity of obtained bicyclic thiazolidine hydantoin. The standard sample shown in Appendix 5 was tested under the following operational conditions:
HPLC Type
Pump: Waters 600E
Detector: Waters 2996 Photodiode Array Detector
Autosampler: Waters 717 plus
Mobile phase: 1% TEA, pH7.5/MeOH=40/60
Flow rate: 1.0 mL/min
Column: Inertsil 5 ODS-80A, 3.2*250-mm
Column Oven: 40 C
Wavelength: 254 nm
RT: peak at 1 min indicates a retention time for methanol.
RT: peak at 13.797 min indicates a retention time for bicyclic thiazolidine hydantoin (R¹=Ph, R²=PhCH₂).
RT: retention time

According to the examples and experimental data, the method for synthesizing chiral bicyclic thiazolidine hydantoin can synthesize bicyclic thiazolidine hydantoin by two cycloadditions within only a singular reacting chamber without isolating the intermediate (so-called one-pot operation) and has high producing rate over and about 80%. Therefore, operational procedures are simplified in a convenient way and operational time is decreased, whereby this method has excellent economic benefits. Additionally, the solid molecular sieves increase the crystallization degree of bicyclic thiazolidine hydantoin. Therefore, the two main drawbacks of the conventional method for manufacturing bicyclic thiazolidine hydantoin are eliminated in the present invention.

Although the invention has been explained in relation to its preferred embodiment, many other possible modifications and variations can be made without departing from the spirit and scope of the invention as hereinafter claimed.

1. A method for synthesizing chiral bicyclic thiazolidine hydantoin, the method taking L-(+)-Cysteine, an aldehyde, an isocyanate as reactants with additive solid molecular sieves to synthesize chiral bicyclic thiazolidine hydantoin and performing in accordance with the following chemical equation: 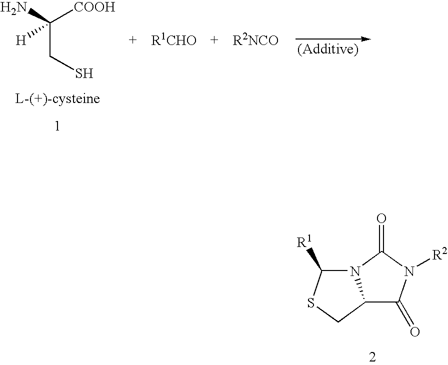
wherein R¹ and R² are selected from the group comprising a hydrogen, phenyl, benzyl, alkyl group containing 1 to 5 carbon atoms, aryl alkyl group in which the alkyl containing 1 to 5 carbon atoms.

2. The method as claimed in claim 1, wherein the isocyanate is benzylisocyanate.

3. The method as claimed in claim 2, the method comprising following operational acts of:
mixing L-(+)-Cysteine, aldehyde, an organic alkali, an organic alcohol solvent to carry out a first cycloaddition to compose a solution and to generate white intermediate, wherein the organic alcohol contains 1 to 5 carbon atoms;
extracting the alcohol solvent;
adding the solid molecular sieves, benzylisocyanate and a ketone solvent to mix well in the solution to carry out a second cycloaddition;
extracting the ketone solvent;
adding ether solvent and an inorganic acid to mix well in the solution;
placing the solution to separate the solution into an upper ether layer and a lower aqueous layer with deposited solid molecular sieves;
removing the ether solvent;
adding an alcohol solvent to enforce crystallization of bicyclic thiazolidine hydantoin in the form of a white solid, wherein the alcohol contains 1 to 4 carbons;
extracting the alcohol solvent; and
drying the crystallization to obtain a final bicyclic thiazolidine hydantoin.

4. The method as claimed in claim 3, wherein the organic alcohol solvent is an organic alcohol-water solvent in a ratio of water:organic alcohol=1:1.

5. The method as claimed in claim 3, wherein the ketone solvent contains ketone having 2-5 carbons.

6. The method as claimed in claim3, wherein the organic alkali is sodium acetate.

7. The method as claimed in claim 3, wherein the organic alkali is potassium acetate.

8. The method as claimed in claim 3, wherein the solid molecular sieves are in the form of particles having 3 Å-5 Å bore diameters.

9. The method as claimed in claim 3, wherein the ether solvent is diethyl ether.

10. The method as claimed in claim 3, wherein the reaction temperature range is within 25 to 50° C.

11. The method as claimed in claim 2, the method comprising the following operational acts of:
mixing L-(+)-Cysteine, an aldehyde, an organic alkali, an organic alcohol solvent to carry out a first cycloaddition to compose a solution and to generate white intermediate, wherein the organic alcohol contains 1 to 5 carbons;
extracting the alcohol solvent;
adding the solid molecular sieves, benzylisocyanate and a ketone solvent to mix well in the solution to carry out a second cycloaddition;
extracting the ketone solvent;
adding ester solvent and an inorganic acid to mix well in the solution;
placing the solution to separate the solution into an upper ester layer and a lower aqueous with deposited solid molecular sieves;
removing the ester solvent;
adding an alcohol solvent to enforce crystallization of bicyclic thiazolidine hydantoin in the form of a white solid, wherein the alcohol contains 1 to 4 carbons;
extracting the alcohol solvent; and
drying the crystallization to obtain a final bicyclic thiazolidine hydantoin.

12. The method as claimed in claim 11, wherein the organic alcohol solvent is an organic alcohol-water solvent in a ratio of water:organic alcohol=1:1.

13. The method as claimed in claim 11, wherein the ketone solvent contains ketone having 2-5 carbons.

14. The method as claimed in claim 11, wherein the organic alkali is sodium acetate.

15. The method as claimed in claim 11, wherein the organic alkali is potassium acetate.

16. The method as claimed in claim 11, wherein the solid molecular sieves are in the form of particles having 3 Å-5 Å bore diameters.

17. The method as claimed in claim 11, wherein the ester solvent is made of ester selected from the group consisting of methyl formate, ethyl formate, methyl acetate, ethyl acetate, and propyl acetate.

18. The method as claimed in claim 11, wherein reaction temperature range is within 25 to 50° C.
